(19) United States
(12) Patent Application Publication
(10) Pub. No.: US 2013/0172558 A1
(43) Pub. Date: Jul. 4, 2013

(54) NAPROXEN-BASED CHIRAL COMPOUNDS AND LIQUID CRYSTAL DISPLAY APPLICATIONS

(71) Applicants: Yongqiang Zhang, Longmont, CO (US); Kundalika More, Denver, CO (US); William Thurmes, Longmont, CO (US)

(72) Inventors: Yongqiang Zhang, Longmont, CO (US); Kundalika More, Denver, CO (US); William Thurmes, Longmont, CO (US)

(21) Appl. No.: 13/343,528

(22) Filed: Jan. 4, 2012

(51) Int. Cl. C07D 239/28; C07D 285/12; C07C 69/616; C07D 213/55; C07C 69/78; C07C 69/734

(52) U.S. Cl. 544/335; 560/80; 560/56; 560/100; 546/342; 548/136

(57) ABSTRACT

Naproxen-based ferroelectric liquid crystal (FLC) or nematic liquid crystal chiral compounds, which can be used in liquid crystal compositions useful for electro-optical and display device applications. The liquid crystal can be a ferroelectric liquid crystal. Also provided is a display device that includes the Naproxen-based ferroelectric liquid crystal (FLC) or nematic liquid crystal chiral compound. Also provided is a method of preparing a liquid crystal display device on silicon that includes incorporating the Naproxen-based ferroelectric liquid crystal (FLC) or nematic liquid crystal chiral compound, into a liquid crystal display on silicon by disposing the compound in a liquid crystal, or the liquid crystal, onto a silicon surface.

### BACKGROUND

Ferroelectric liquid crystal (FLC) displays offer great advantages in terms of quick response, the time needed for a change in orientation of the ferroelectric liquid crystal array being much shorter than the time needed for the change in a typical nematic liquid crystal. One great advantage of this quick response is that sequential coloring of pixels is possible at a refresh rate that facilitates color fusion, that is, the appearance to the human eye of the pixel as a single color rather than as rapidly sequentially changing colors. Sequential color also enables a higher resolution for a particular pixel size, as a single pixel can display all colors, rather than requiring three pixels, red, green and blue, to display a full color spectrum.

In formulating new ferroelectric liquid crystal (FLC) mixtures, there are some properties that are generally viewed as broadly desirable, and others that are adjusted depending on the target application. Micron's Display division produces liquid crystal on silicon (LCOS) displays, in which light is reflected within the cell, requiring very thin cells. Some examples of FLC mixture properties that are almost always desired for use in these thin cells are good alignment, low viscosity, high tilt angle, a high SmA to SmC transition, a low crystallization temperature, and an I-N-SmA-SmC phase progression. Examples of properties more dependent on the target application include the magnitude of the birefringence and the magnitude of the polarization.

A variety of patents describe FLC mixtures, displays, and devices. For example, see U.S. Pat. No. 4,325,830, U.S. Pat. No. 4,565,425, U.S. Pat. No. 4,704,227, DE 4,102,837, U.S. Pat. No. 4,325,850, U.S. Pat. No. 5,104,569, U.S. Pat. No. 5,100,577, U.S. Pat. No. 5,683,623, U.S. Pat. No. 6,059,994, U.S. Pat. No. 6,174,457, U.S. Pat. No. 4,776,973, U.S. Pat. No. 5,858,272, U.S. Pat. No. 4,200,580, U.S. Pat. No. 4,344,856, U.S. Pat. No. 4,348,324, U.S. Pat. No. 5,849,216, U.S. Pat. No. 4,450,094, U.S. Pat. No. 4,709,030, U.S. Pat. No. 5,030,383, and U.S. Pat. No. 5,653,913, all of which are incorporated by reference herein in their entireties.

### BRIEF DESCRIPTION OF THE DRAWINGS

Embodiments may be best understood by referring to the following description and accompanying drawings which illustrate such embodiments. The numbering scheme for the Figures included herein are such that the leading number for a given reference number in a Figure is associated with the number of the Figure. In the drawings:

FIG. 1 illustrates an example of a liquid crystal image system, according to an embodiment.

FIG. 2 illustrates another example of a liquid crystal image system, according to an embodiment.

FIG. 3 illustrates an example of a liquid crystal device, according to an embodiment.

### DESCRIPTION

Reference will now be made in detail to certain claims, examples of which are illustrated in the accompanying structures and formulas. While the invention will be described in conjunction with the enumerated claims, it will be understood that they are not intended to limit the invention as claimed. On the contrary, the invention is intended to cover all alternatives, modifications, and equivalents, which may be included within the scope of the invention as defined by the claims.

References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one of ordinary skill in the art to affect such feature, structure, or characteristic in connection with other embodiments whether or not explicitly described.

In various embodiments, a ferroelectric liquid crystal mixture that includes a naproxen-based compound is provided, as described herein. The incorporation of a naproxen-based compound into FLC molecules can introduce bookshelf layer structures, as well as can adjust the pitch and spontaneous polarization of the FLC materials when they are used as FLC chiral dopants. FLC microdisplays with bookshelf structures are significantly advantageous over the current commercial ones with chevron structures which lead to the undesired zigzag defect and make optical bistability difficult to realize. A bistable device has the following advantages: stable memory performance, high contrast ratio, wide angle view, high speed response, and/or lower power consumption.

Some embodiments are directed toward naproxen-based ferroelectric liquid crystal (FLC) or nematic liquid crystal chiral compounds, which can be used in liquid crystal compositions useful for electro-optical and display device applications. The naproxen-based ferroelectric liquid crystal (FLC) or nematic liquid crystal chiral compounds include compounds of formula (I):

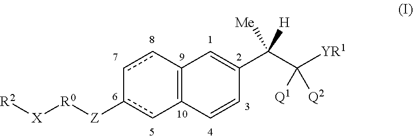

wherein,

R¹ is (C₂-C₂₀)alkyl, (C₂-C₂₀)alkenyl, or (C₂-C₂₀)alkynyl;

R² is (C₂-C₂₀)alkyl, (C₂-C₂₀)alkenyl, or (C₂-C₂₀)alkynyl;

Q¹ and Q² are each hydrogen, or together Q¹ and Q² are oxo (═O) or thioxo (═S);

Z is O—C(═O), C(═O)O, CH₂O, CH₂CH₂, OCH₂, or is absent;

X is oxy (—O—), O—C(═O), C(═O)O, CH₂O, CH₂CH₂, OCH₂, or is absent;

Y is oxy (—O—) or thio (—S—);

R⁰ is an aromatic ring system, a cyclic ring system, a hetero aromatic ring system, a hetero cyclic ring system, or a combination thereof;

each bond represented by - - - - - - is independently optionally present;

any one or more of the aromatic ring system, cyclic ring system, hetero aromatic ring system, hetero cyclic ring system, or combination thereof of R⁰ is optionally substituted on carbon with one or more halo;

any one or more of the (C₂-C₂₀)alkyl, (C₂-C₂₀)alkenyl, or (C₂-C₂₀)alkynyl of R¹ and R² is optionally substituted on carbon with one or more halo; and

any one or more of the (C₂-C₂₀)alkyl, (C₂-C₂₀)alkenyl, or (C₂-C₂₀)alkynyl of R¹ and R² is optionally interrupted with one or more oxy (—O—), thio (—S—), Si(R_{A})(R_{B}) or Ge(R_{A}R_{B}), wherein each R_{A} and R_{B} is independently (C₁-C₆)alkyl or (C₁-C₆)alkenyl.

Some embodiments are also directed to a liquid crystal that includes the compound of formula (I). The liquid crystal can be a ferroelectric liquid crystal.

Some embodiments are also directed to a display device that includes the compound of formula (I) or the liquid crystal described herein. The display device can have at least one of stable memory performance, high contrast ratio, wide angle view, high speed response, and lower power consumption, relative to a comparable device not including a compound of formula (I). The display device can include a ferroelectric liquid crystal composition. The display device can be a cell phone, a smart phone, a tablet, or a computer display screen.

Some embodiments are also directed to a method of preparing a liquid crystal display device on silicon that includes incorporating the compound of formula (I) or the liquid crystal described herein, into a liquid crystal display on silicon by disposing the compound in a liquid crystal, or the liquid crystal, onto a silicon surface.

Some embodiments relate to ferroelectric liquid crystal mixtures that include a naproxen-based compound. When describing the ferroelectric liquid crystal mixtures, naproxen-based compounds, or electronic devices that include the same, the following terms have the following meanings, unless otherwise indicated.

### DEFINITIONS

Unless stated otherwise, the following terms and phrases as used herein are intended to have the following meanings:

“Chiral” refers to asymmetrical molecules that are mirror images of one another, i.e., they are related like left and right hands. Such molecules have one or more chiral centers, and are characterized by optical activity. It will be appreciated by those of ordinary skill in the art that compounds having a chiral center may exist in and be isolated in optically active and racemic forms. Some compounds may exhibit polymorphism. It is to be understood that any racemic, optically-active, polymorphic, or stereoisomeric form, or mixtures thereof, of a compound described herein, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine anti-fungal or anti-bacterial activity using the standard tests described herein, or using other similar tests which are well known in the art.

One diastereomer of a compound disclosed herein may display superior activity compared with the other. When required, separation of the racemic material can be achieved by HPLC using a chiral column or by a resolution using a resolving agent such as camphonic chloride as in Tucker et al., J. Med. Chem., 37:2437 (1994). A chiral compound described herein may also be directly synthesized using a chiral catalyst or a chiral ligand, e.g. Huffman et al., J. Org. Chem., 60:1590 (1995).

“Stable compound” and “stable structure” are meant to indicate a compound that is sufficiently robust to survive isolation to a useful degree of purity from a reaction mixture. Only stable compounds are contemplated herein.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. A suitable indicated group includes, e.g., halo.

“Interrupted” is intended to indicate that in between two or more adjacent carbon atoms, and the hydrogen atoms to which they are attached (e.g., methyl (CH₃), methylene (CH₂) or methine (CH)), indicated in the expression using “interrupted” is inserted with a selection from the indicated group(s), provided that the each of the indicated atoms' normal valency is not exceeded, and that the interruption results in a stable compound. Such suitable indicated groups include, e.g., one or more non-peroxide oxy (—O—) and thio (—S—).

“Alkyl” refers to a C₂-C₂₀ or C₁-C₆ hydrocarbon containing normal, secondary, tertiary or cyclic carbon atoms. Examples are methyl, ethyl, 1-propyl, 2-propyl, 1-butyl, 2-methyl-1-propyl (iso-butyl, —CH₂CH(CH₃)₂), 2-butyl (sec-butyl, —CH(CH₃)CH₂CH₃), 2-methyl-2-propyl (tert-butyl, —C(CH₃)₃), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl.

The alkyl can be a monovalent hydrocarbon radical, as described and exemplified above, or it can be a divalent hydrocarbon radical (i.e., alkylene).

The alkyl can optionally be substituted on carbon, e.g., with one or more halo.

“Alkenyl” refers to a C₂-C₂₀ or C₁-C₆ hydrocarbon containing normal, secondary, tertiary or cyclic carbon atoms with at least one site of unsaturation, i.e. a carbon-carbon, sp² double bond. Examples include, but are not limited to: ethylene or vinyl (—CH═CH₂), allyl (—CH₂CH═CH₂), cyclopentenyl (—C₅H₇), and 5-hexenyl (—CH₂ CH₂CH₂CH₂CH═CH₂). The alkenyl can be a monovalent hydrocarbon radical, as described and exemplified above, or it can be a divalent hydrocarbon radical (i.e., alkenylene).

The alkenyl can optionally be substituted on carbon, e.g., with one or more halo.

“Alkynyl” refers to a C₂-C₂₀ or C₁-C₆ hydrocarbon containing normal, secondary, tertiary or cyclic carbon atoms with at least one site of unsaturation, i.e. a carbon-carbon, sp triple bond. Examples include, but are not limited to: ethylyne (—CH≡CH), propylyne (—CH₂CH≡CH₂), and 5-hexynyl (—CH₂ CH₂CH₂CH₂CH≡CH₂). The alkynyl can be a monovalent hydrocarbon radical, as described and exemplified above, or it can be a divalent hydrocarbon radical (i.e., alkynylene).

The alkynyl can optionally be substituted on carbon, e.g., with one or more halo.

The term “aryl” refers to an unsaturated aromatic carbocyclic group of from 6 to 20 carbon atoms having a single ring (e.g., phenyl), multiple rings (e.g., biphenyl), or multiple condensed (fused) rings, wherein at least one ring is aromatic (e.g., naphthyl, dihydrophenanthrenyl, fluorenyl, or anthryl). Preferred aryls include phenyl, biphenyl, naphthyl and the like. The aryl can optionally be a divalent radical, thereby providing an arylene.

The aryl can optionally be substituted on carbon, e.g., with one or more halo.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted on carbon, e.g., with one or more halo.

The cycloalkyl can optionally be at least partially unsaturated, thereby providing a cycloalkenyl. Additionally, the cycloalkyl can optionally be a divalent radical, thereby providing a cycloalkylene.

The term “halo” refers to fluoro, chloro, bromo, and iodo. Similarly, the term “halogen” refers to fluorine, chlorine, bromine, and iodine.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted. The heteroaryl can optionally be a divalent radical, thereby providing a heteroarylene.

Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl.

In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted on carbon with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyannato, sulfamoyl, sulfinamoyl, sulfino, sulfo, sulfoamino, thiosulfo, NR^{x}R^{y} and/or COOR^{x}, wherein each Rx and R^{y} are independently H, alkyl, alkenyl, aryl, heteroaryl, heterocycle, cycloalkyl or hydroxy.

The term “heterocycle” refers to a saturated or partially unsaturated ring system, containing at least one heteroatom selected from the group oxygen, nitrogen, and sulfur, and optionally substituted with alkyl or C(═O)OR^{b}, wherein R^{b} is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine. The heterocycle can optionally be a divalent radical, thereby providing a heterocyclene.

The heterocycle can optionally be substituted on carbon with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, acetamido, acetoxy, acetyl, benzamido, benzenesulfinyl, benzenesulfonamido, benzenesulfonyl, benzenesulfonylamino, benzoyl, benzoylamino, benzoyloxy, benzyl, benzyloxy, benzyloxycarbonyl, benzylthio, carbamoyl, carbamate, isocyannato, sulfamoyl, sulfinamoyl, sulfino, sulfo, sulfoamino, thiosulfo, NR^{x}R^{y} and/or COOR^{x}, wherein each R^{x} and R^{y} are independently H, alkyl, alkenyl, aryl, heteroaryl, heterocycle, cycloalkyl or hydroxy.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. In one specific embodiment, the nitrogen heterocycle can be 3-methyl-5,6-dihydro-4H-pyrazino[3,2,1-jk]carbazol-3-ium iodide.

Another class of heterocyclics is known as “crown compounds” which refers to a specific class of heterocyclic compounds having one or more repeating units of the formula [—(CH₂-)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “oxy” refers to —O—.

The term “thio” refers to —S—.

The term “thioxo” refers to (═S).

The term “oxo” refers to (═O).

As to any of the above groups, which contain one or more substituents, it is understood, of course, that such groups do not contain any substitution or substitution patterns which are sterically impractical and/or synthetically non-feasible. In addition, the compounds include all stereochemical isomers arising from the substitution of these compounds.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of organic chemistry understands that the total number of such substituents is reasonably limited by the desired properties of the compound intended. Such properties include, by of example and not limitation, physical properties such as molecular weight, solubility or log P, application properties, and practical properties such as ease of synthesis.

Recursive substituents are an intended aspect. One of ordinary skill in the art of organic chemistry understands the versatility of such substituents. To the degree that recursive substituents are present in a claim, the total number will be determined as set forth above.

Obviously, numerous modifications and variations are possible in light of the above teachings. It is therefore to be understood that within the scope of the appended claims, the disclosed subject matter may be practiced otherwise than as specifically described herein.

Specific ranges, values, and embodiments provided below are for illustration purposes only and do not otherwise limit the scope of the disclosed subject matter, as defined by the claims.

### Specific Ranges, Values, and Embodiments

For the compound of formula (I):
R¹ can be a non-racemic chiral group.
R¹ can be a non-racemic chiral group, selected from:

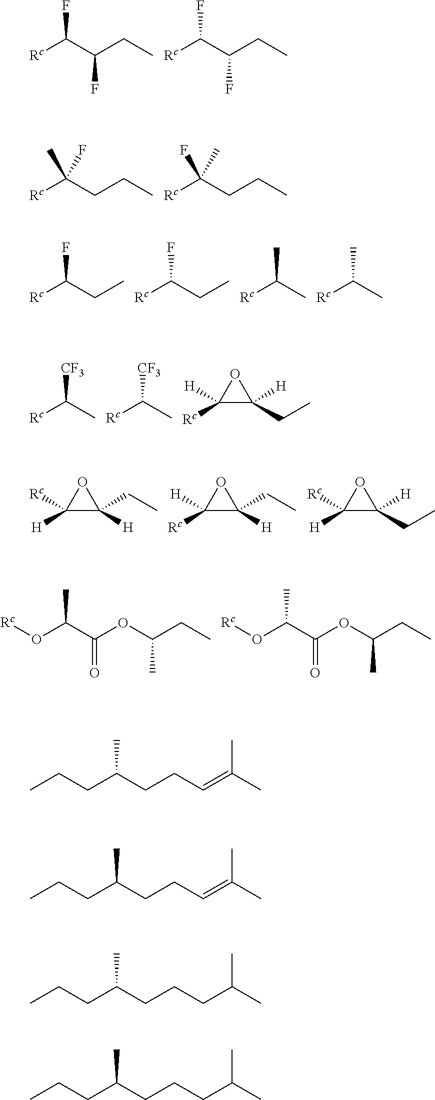

wherein,
Rc is (C₁-C₁₅)alkyl, (C₁-C₁₅)alkenyl, or (C₁-C₁₅)alkynyl, optionally substituted on carbon with one or more halo, and optionally interrupted with one or more oxy (—O—), thio (—S—), Si(R_{A})(R_{B}) or Ge(R_{A}R_{B}), wherein each R_{A} and R_{B} is independently (C₁-C₆)alkyl or (C₁-C₆)alkenyl.

R¹ can be selected from:

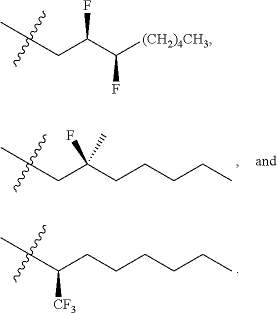

R² can be a non-racemic chiral group.

R² can be a non-racemic chiral group, selected from:

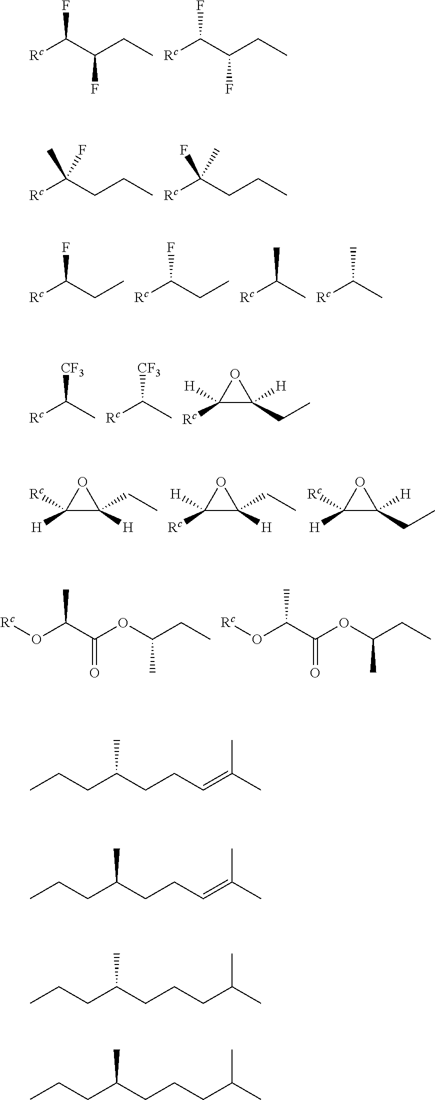

wherein,
Rc is (C₁-C₁₅)alkyl, (C₁-C₁₅)alkenyl, or (C₁-C₁₅)alkynyl, optionally substituted on carbon with one or more halo, and optionally interrupted with one or more oxy (—O—), thio (—S—), Si(R_{A})(R_{B}) or Ge(R_{A}R_{B}), wherein each R_{A} and R_{B} is independently (C₁-C₆)alkyl or (C₁-C₆)alkenyl.

R² can be selected from:

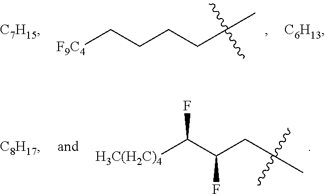

R⁰ can be —(R^{x})ₙ-(A)ₐ-(Ph¹)ₗ-(B)_{b}-(Ph²)ₘ-

wherein,
R^{x} is cyclohexyl or cyclohexenyl, optionally substituted on carbon with one or more halo or cyano, and one or two of the methylene (CH₂) groups of the cyclohexyl or cyclohexenyl is optionally independently replaced with oxy (—O—);
n is 0 or 1;
A is oxy (—O—), thio (—S—), CH₂O, OCH₂, CH₂S, SCH₂, CH₂OCO, CH₂CO₂, CH₂CH₂, COS, CSO, COO, OCO, a single bond (absent), a double bond, or a triple bond;
a is 0 or 1;
Ph¹ is 1,4-phenyl, optionally substituted on carbon with one or more halo, and one or two of the ring carbons are optionally independently replaced with nitrogen (N) or a thiadiazole ring;
l is 0 or 1;
B is oxy (—O—), thio (—S—), CH₂O, OCH₂, CH₂S, SCH₂, CH₂OCO, CH₂CO₂, CH₂CH₂, COS, CSO, COO, OCO, a single bond (absent), a double bond, or a triple bond;
b is 0 or 1;
Ph² is 1,4-phenyl, optionally substituted on carbon with one or more halo, and one or two of the ring carbons are optionally independently replaced with nitrogen (N) or a thiadiazole ring;
m is 0 or 1;
when m is 0, then b is also 0; and
at least one of n, a, 1, b and m is 1.

R⁰ can be an aromatic ring system or a cyclic ring system.

R⁰ can be phenyl, 3-fluorophenyl, biphenyl, 2,3-difluorophenyl, cyclohexyl, 2-phenylpyrimidine, 2-phenylthiadiazo, or 2-phenylpyridine.

The bond represented - - - - - - by between carbon atoms 5 and 6, and the bond represented by - - - - - - between carbon atoms 7 and 8, can each be absent.

The compound of claim 1, wherein the bond represented by - - - - - - between carbon atoms 5 and 6, and the bond represented by - - - - - - between carbon atoms 7 and 8, can each be present.

Q¹ and Q² together can be oxo (═O).

Z can be C(═O)O, CH₂CH₂, absent, or OCH₂.

X can be oxy (—O—), absent or CH₂O.

X can be oxy (—O—).

### Specific Compounds

Specific compounds include the compounds below, which are also further illustrated in Table A.

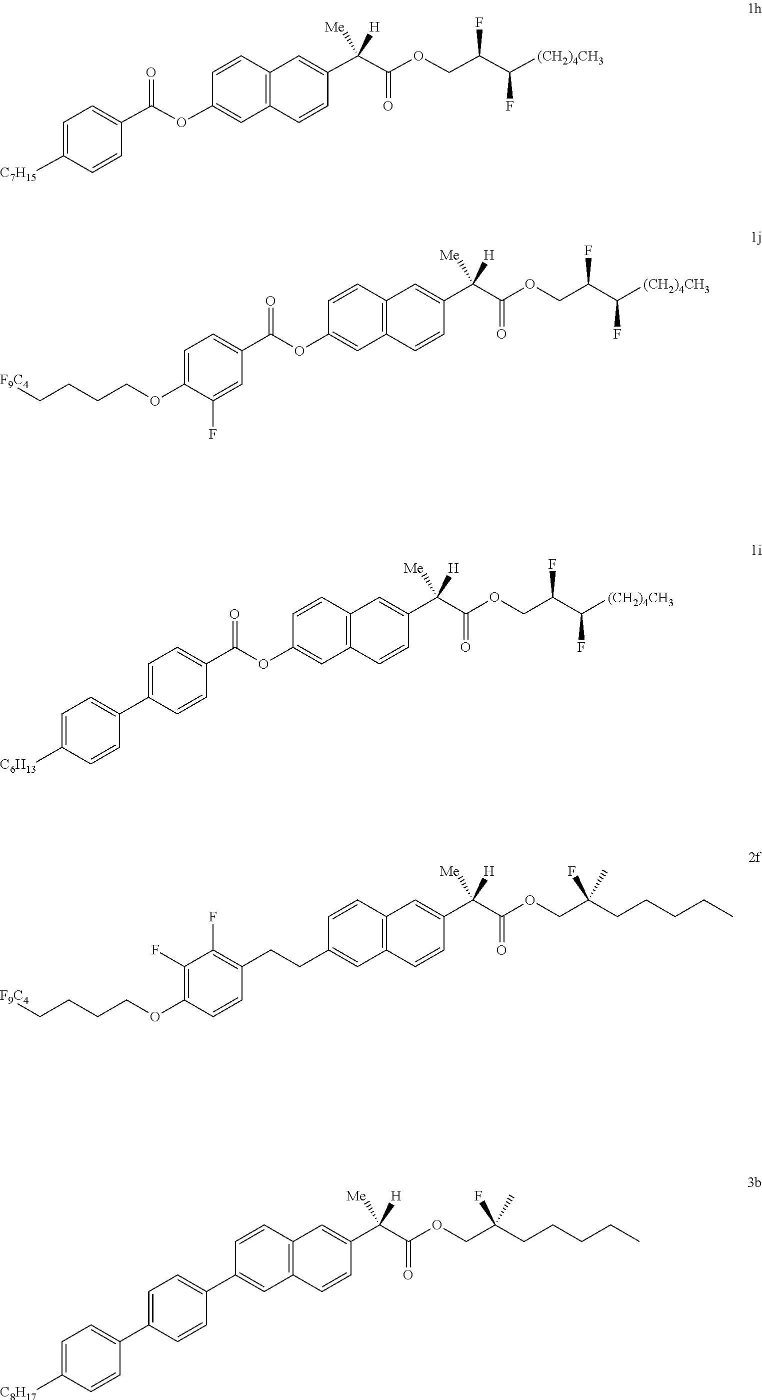

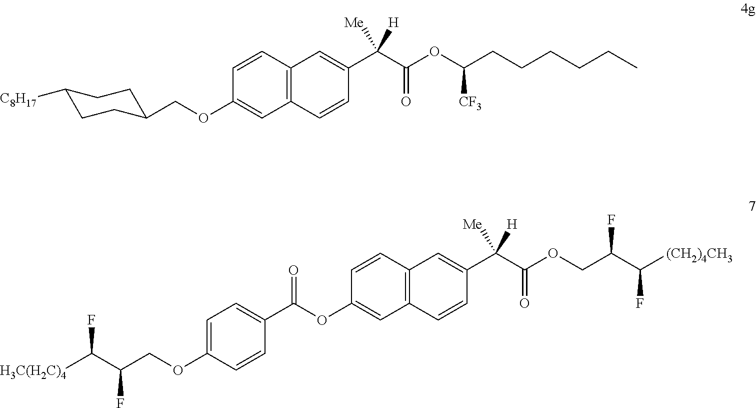

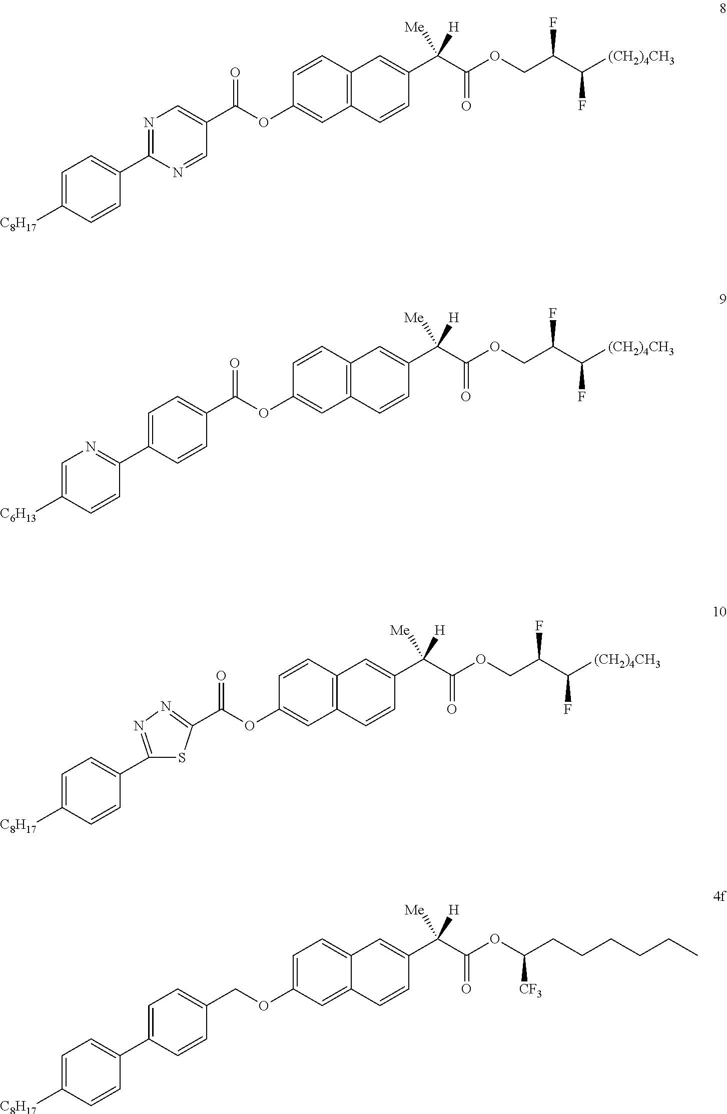

### Methods of Making the Compounds

The compounds described herein can be prepared by any of the applicable techniques of organic synthesis. Many such techniques are well known in the art. However, many of the known techniques are elaborated in Compendium of Organic Synthetic Methods (John Wiley & Sons, New York) Vol. 1, Ian T. Harrison and Shuyen Harrison (1971); Vol. 2, Ian T. Harrison and Shuyen Harrison (1974); Vol. 3, Louis S. Hegedus and Leroy Wade (1977); Vol. 4, Leroy G. Wade Jr., (1980); Vol. 5, Leroy G. Wade Jr. (1984); and Vol. 6, Michael B. Smith; as well as March, J., Advanced Organic Chemistry, 3rd Edition, John Wiley & Sons, New York (1985); Comprehensive Organic Synthesis. Selectivity, Strategy & Efficiency in Modern Organic Chemistry, In 9 Volumes, Barry M. Trost, Editor-in-Chief, Pergamon Press, New York (1993); Advanced Organic Chemistry, Part B: Reactions and Synthesis, 4th Ed.; Carey and Sundberg; Kluwer Academic/Plenum Publishers: New York (2001); Advanced Organic Chemistry, Reactions, Mechanisms, and Structure, 2nd Edition, March, McGraw Hill (1977); Protecting Groups in Organic Synthesis, 2nd Edition, Greene, T. W., and Wutz, P. G. M., John Wiley & Sons, New York (1991); and Comprehensive Organic Transformations, 2nd Edition, Larock, R. C., John Wiley & Sons, New York (1999). Representative synthetic procedures are illustrated in the Examples herein.

It is appreciated that those of ordinary skill in synthetic organic chemistry understand that reagents are typically referred to by the chemical names that they bear or formulae that represent their structures prior to addition to a chemical reaction mixture, even though the chemical species actually present in the reaction mixture or involved in the reaction may be otherwise. While a compound may undergo conversion to a compound bearing a different name or represented by a different formula prior to or during a specified reaction step, reference to these compounds by their original name or formula is acceptable and is well-understood by those of ordinary skill in the art of organic chemistry.

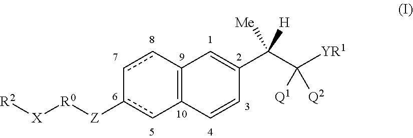

TABLE A

| | | | | | bond | bond | | | |
|---|---|---|---|---|---|---|---|---|---|
| | | | | | represented | represented | | | |
| | | | | | by - | by - | | | |
| | | | | | between | between | | | |
| | | | | | carbon | carbon | | | |
| | | | | | atoms 5 and | atoms 7 and | | | |
| Compound | R² | X | R⁰ | Z | 6 | 8 | Q¹ and Q² | Y | R¹ |
| 1h | C₇H₁₅ | Absent | Ph 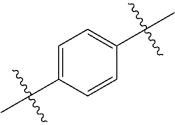 [SMILES: *c1ccc(*)cc1] | C(═O)O | Present | Present | Together are oxo (═O) | O | 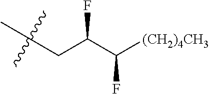 |
| 1j | 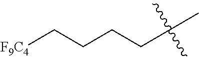 | O | 3-fluorophenyl 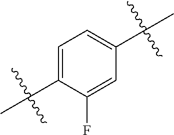 [SMILES: *c1ccc(*)c(F)c1] | C(═O)O | Present | Present | Together are oxo (═O) | O | 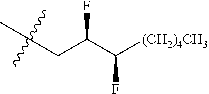 |
| 1i | C₆H₁₃ | Absent | Biphenyl 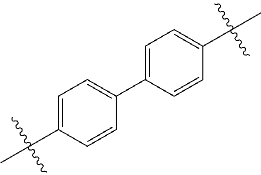 [SMILES: *c1ccc(-c2ccc(*)cc2)cc1] | C(═O)O | Present | Present | Together are oxo (═O) | O | 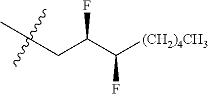 |
| 2f | 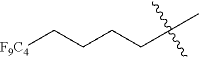 | O | 2,3-difluorophenyl 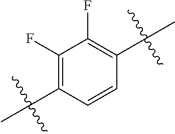 [SMILES: *c1ccc(*)c(F)c1F] | CH₂CH₂ | Present | Present | Together are oxo (═O) | O | 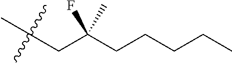 [SMILES: *C[C@@](C)(F)CCCCC] |
| 3b | C₈H₁₇ | Absent | Biphenyl 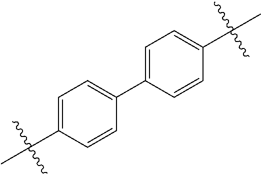 [SMILES: *c1ccc(-c2ccc(*)cc2)cc1] | Absent | Present | Present | Together are oxo (═O) | O | 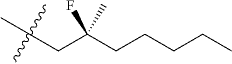 [SMILES: *C[C@@](C)(F)CCCCC] |
| 4g | C₈H₁₇ | Absent | Cyclohexyl 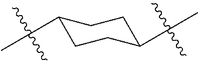 [SMILES: *C1CCC(*)CC1] | OCH₂ | Present | Present | Together are oxo (═O) | O | 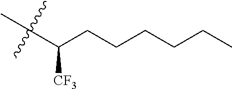 [SMILES: *[C@@H](CCCCCC)C(F)(F)F] |
| 7 | 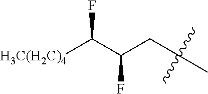 | O | Ph 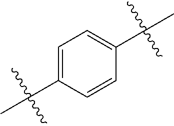 [SMILES: *c1ccc(*)cc1] | C(═O)O | Present | Present | Together are oxo (═O) | O | 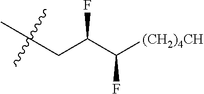 |
| 8 | C₈H₁₇ | Absent | 2-phenylpyrimidine 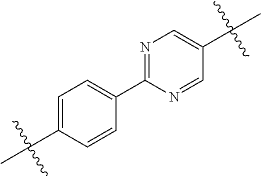 [SMILES: *c1ccc(-c2ncc(*)cn2)cc1] | C(═O)O | Present | Present | Together are oxo (═O) | O | 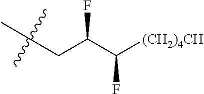 |
| 9 | C₆H₁₃ | Absent | 2-phenylpyridine 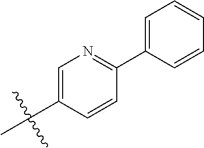 [SMILES: *c1ccc(-c2ccccc2)nc1] | C(═O)O | Present | Present | Together are oxo (═O) | O | 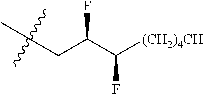 |
| 10 | C₈H₁₇ | Absent | 2-phenylthiadiazo 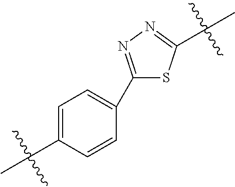 [SMILES: *c1ccc(-c2nnc(*)s2)cc1] | C(═O)O | Present | Present | Together are oxo (═O) | O | 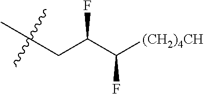 |
| 4f | C₈H₁₇ | Absent | Biphenyl 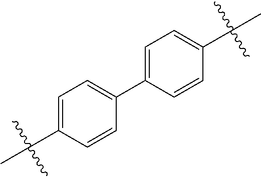 [SMILES: *c1ccc(-c2ccc(*)cc2)cc1] | CH₂O | Present | Present | Together are oxo (═O) | O | 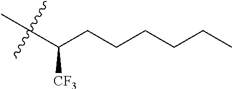 [SMILES: *[C@@H](CCCCCC)C(F)(F)F] |

### Ferroelectric Liquid Crystal Compositions and Devices

In various embodiments, what is provided is a ferroelectric liquid crystal mixture that includes a compound as described herein. The incorporation of a naproxen moiety into FLC molecules can introduce bookshelf layer structures, as well as can adjust the pitch and spontaneous polarization of the FLC materials when they are used as FLC chiral dopants. FLC microdisplays with bookshelf structures are significantly advantageous over the current commercial ones with chevron structures which lead to the undesired zigzag defect and make optical bistability difficult to realize. A bistable device has the following exemplary advantages: stable memory performance, high contrast ratio, wide angle view, high speed response, and/or lower power consumption.

FIGS. 1-3 illustrate some examples of liquid crystal display devices that include liquid crystalline compositions of the present disclosure. FIG. 1 shows a liquid crystal image system 100. In one example, the liquid crystal image system 100 includes a projector system. In one example, the liquid crystal image system 100 includes a screen display system.

The liquid crystal image system 100 includes a light source 110 that provides a light beam 102. The light beam 102 may include a broad spectrum of colors. In one example, only a single color is generated by the light source 110. In one example, different single colors may be provided in rapid succession. In such a configuration, multiple successive single colors are perceived together by a user to form various color combinations or shades of color. In other examples, multiple light sources 110 each concurrently provide a single color light beam 102 (e.g. red, green, blue), that when combined, are perceived together by a user to form various color combinations or shades of color.

A liquid crystal device 120 is included in the system 100 to interact with the light beam 102 and selectively control amounts of light in a resulting image beam 104. A plurality of layers 122 are shown making up the liquid crystal device 120. Examples of layers 122 in the liquid crystal device 120 include, but are not limited to, polarizing layers, reflective layers, textured layers, and electrode layers. FIG. 1 further shows a liquid crystalline material 124 formed from a ferroelectric liquid crystal mixture as described in the present disclosure.

Ferroelectric liquid crystal mixtures may include a number of desirable properties for use in liquid crystal image systems 100. Examples of ferroelectric liquid crystal mixtures as described exhibit faster switching properties than other nematic liquid crystal materials. Fast switching of individual pixels may enable configurations where rapid successions of individual single colors combine to produce full color on each pixel. Examples of ferroelectric liquid crystal mixtures as described may also exhibit bistable crystalline states. Liquid crystal image systems using bistable ferroelectric liquid crystal mixtures may use less power than other liquid crystal image systems, such as active matrix systems. Once a desired state within the ferroelectric liquid crystal mixture is activated, the bistability of the mixture does not require additional power to maintain the selected state.

In one example, the liquid crystal device 120 includes a semiconductor array. In one example, the semiconductor array includes a silicon array that has been processed to include structure for a number of pixels. Examples of semiconductor arrays may include active matrix liquid crystal configurations or passive matrix liquid crystal configurations. The system 100 shown in FIG. 1 illustrates a reflective liquid crystal device 120, although the disclosed subject matter is not so limited. One example of a reflective liquid crystal device 120 includes a Liquid Crystal on Silicon (LCOS) device. Other examples of liquid crystal devices 120 include transmissive configurations where the light 102 selectively passes through the liquid crystal devices 120 depending on a state of individual pixels in the liquid crystal device 120. In the reflective system 100 shown, a first mirror 112 and a second mirror 114 are included to direct the light 102 towards the liquid crystal device 120. In selected examples, as shown in FIG. 1, the image beam 104 passes through the second mirror 114, and interacts with optics 130 to project an image formed by the liquid crystal device 120.

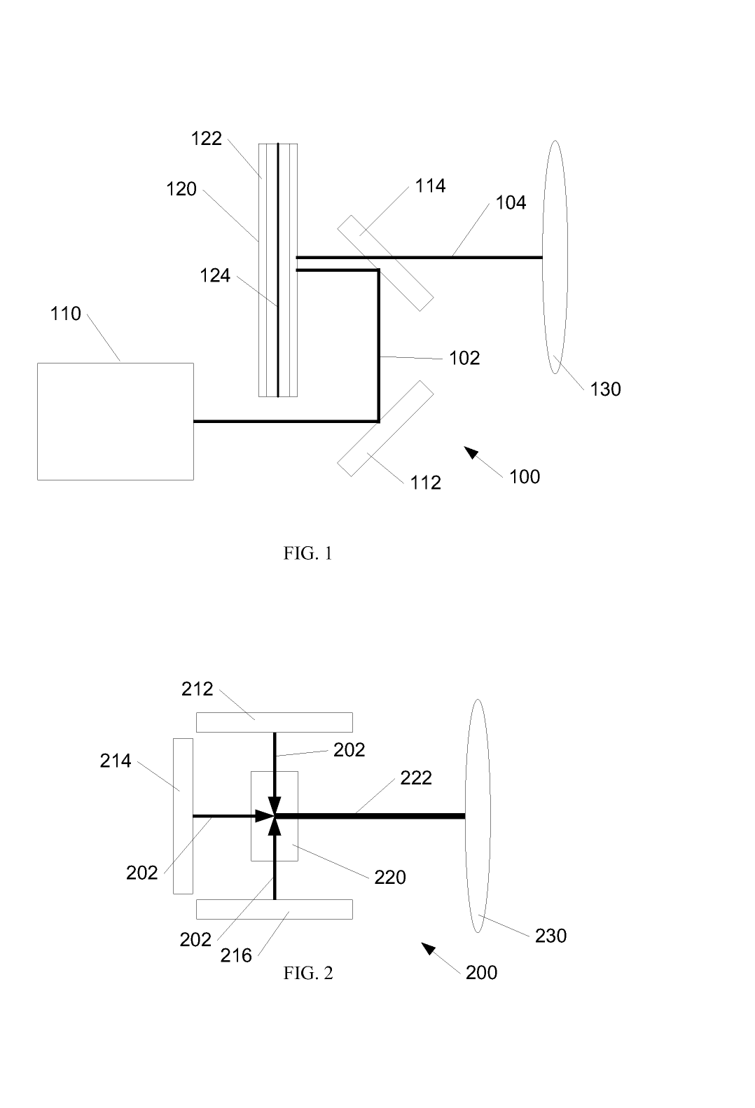

FIG. 2 shows another example of a liquid crystal image system 200. In the system 200, multiple liquid crystal devices are used to produce an image beam 222. FIG. 2 shows a first liquid crystal device 212, a second liquid crystal device 214, and a third liquid crystal device 216. In one example, the liquid crystal devices 212, 214, 216 include elemental colors such as red, green and blue. Component light beams 202 of individual colors are mixed in the liquid crystal image system 200 in a prism 220 to provide the image beam 222.

In one example, one or more of the liquid crystal devices 212, 214, 216 are transmissive. In another example, one or more of the liquid crystal devices 212, 214, 216 are reflective. In one example, multiple light sources (not shown) provide individual component colors such as red, green, and blue for each of the liquid crystal devices 212, 214, 216. In one example, a single light source (not shown) is split using prisms or other splitting devices to divide the light source into component colors that are later re-combined in the prism 120 after modification using the liquid crystal devices 212, 214, 216. Similar to the example illustrated in FIG. 1, in one example, the image beam 222 also interacts with optics 230 to project an image formed by a combined output of the liquid crystal devices 212, 214, 216.

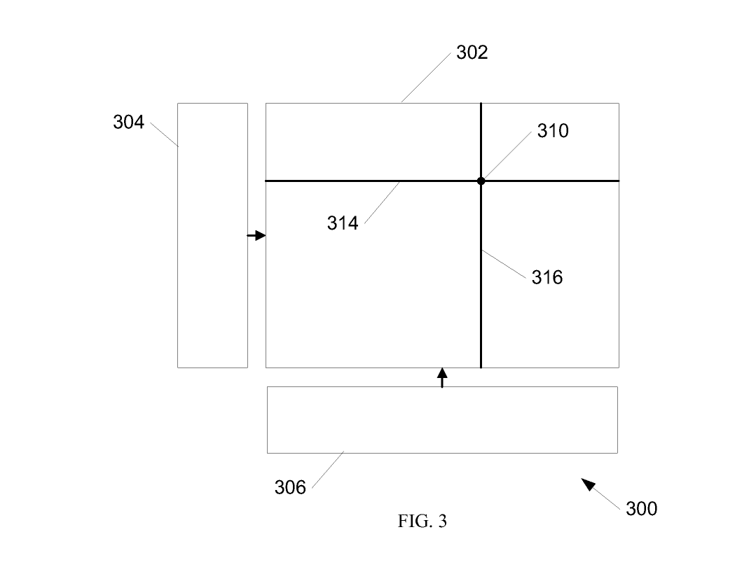

FIG. 3 shows a simplified diagram of a liquid crystal device 300 similar to the liquid crystal devices shown in FIGS. 1 and 2. An array 302 is provided including a number of rows 314 and columns 316. A pixel 310 within the array 302 is defined at an intersection of a given row 314 and column 316. A row controller 304 and a column controller 306 are shown coupled to the array 302. In operation, the row controller activates a desired row 314, and the column controller 306 activates a desired column 316 to select the pixel 310. Progressive activation of successive columns 316 along a given row 314 produce a line within a generated image. Further progressive activation of successive rows and columns generate multiple lines in a progression to generate the full image.

### EXAMPLES

### Synthetic Examples

Naproxen-based FLC or NLC chiral components with two ester linkages at both ends of Naproxen were readily synthesized by the multistep approach as shown below:

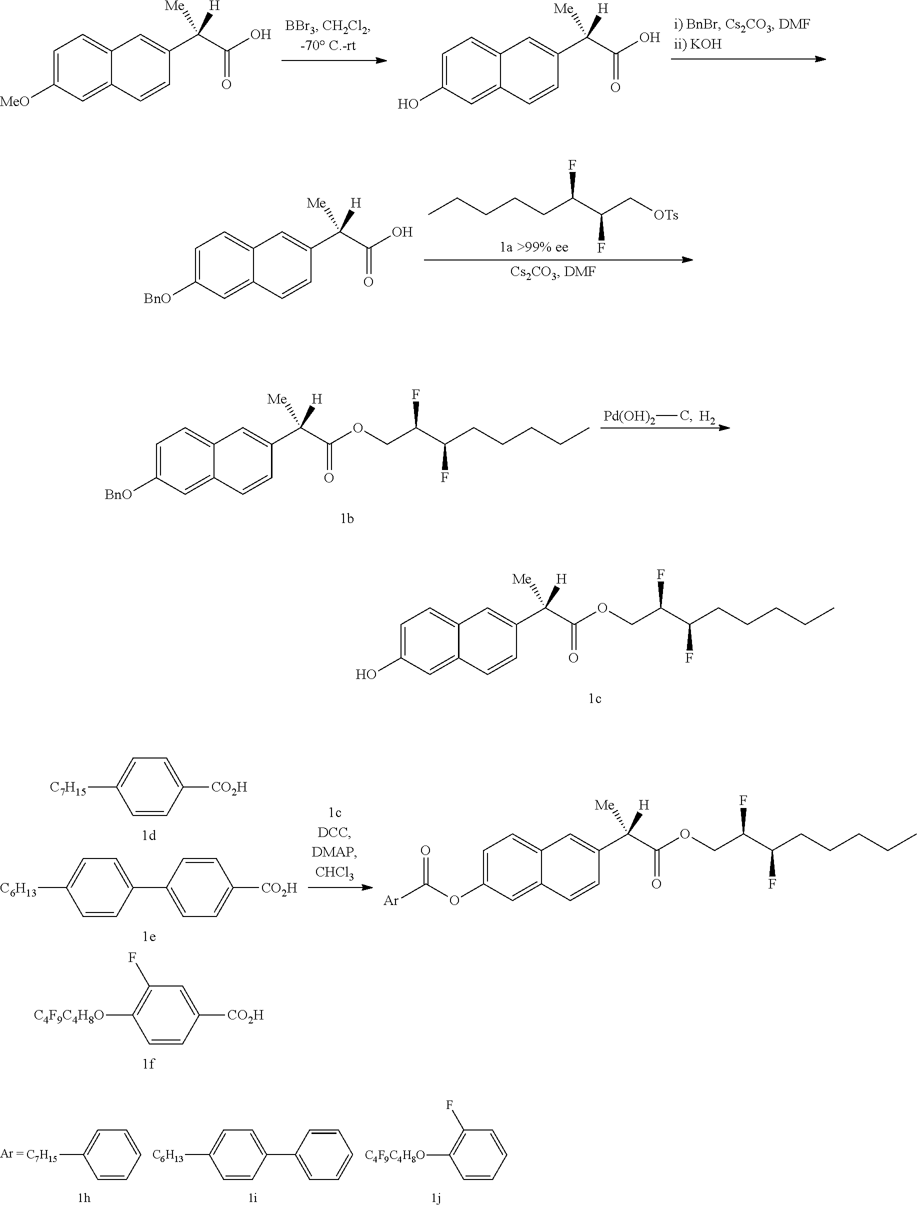

Note that these compounds have been successfully synthesized and well characterized by NMR and LC-MS. A representative Naproxen-based FLC or NLC chiral component with one ester linkage and one CH₂CH₂ linkage at both ends of Naproxen can be readily synthesized by the multistep approach as shown below:

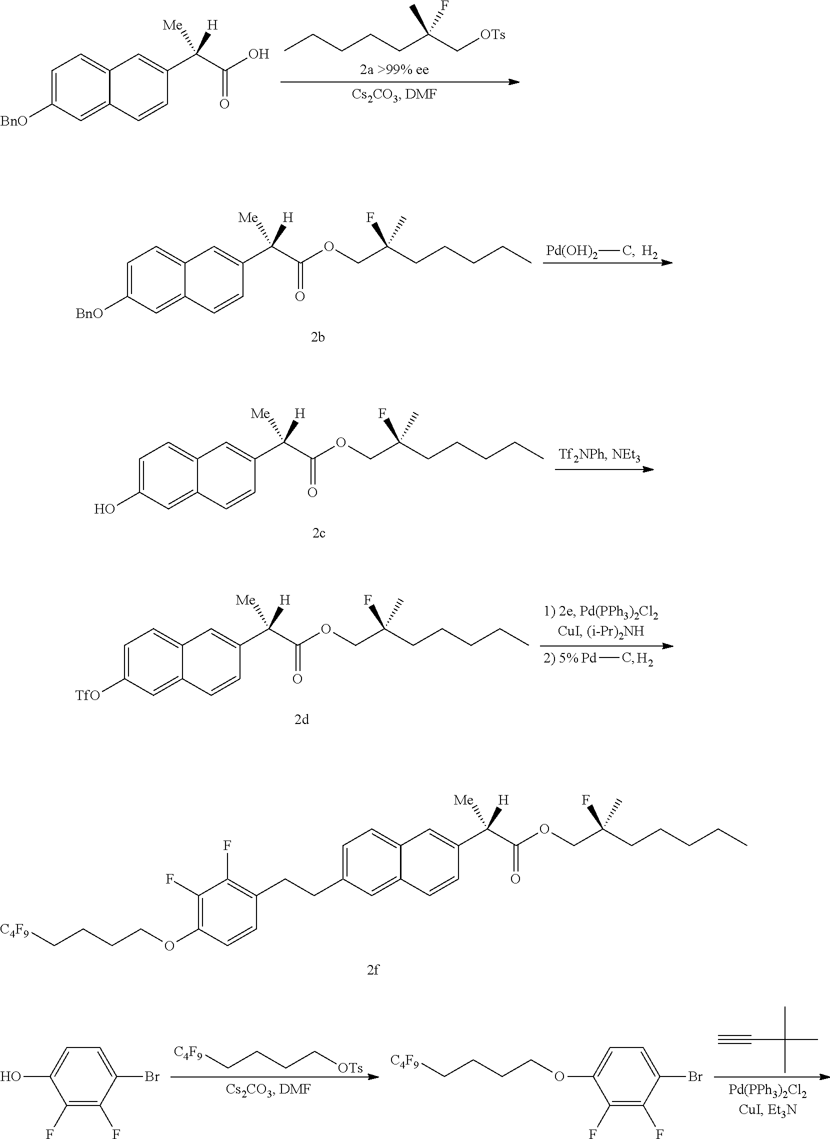

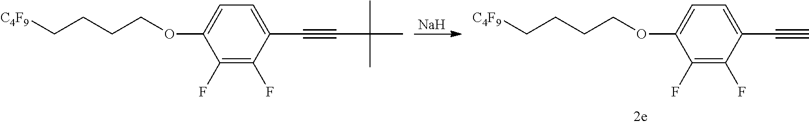

A representative Naproxen-based FLC or NLC chiral component with one ester linkage and one direct bond linkage at both ends of Naproxen can be readily synthesized by the multistep approach as shown below:

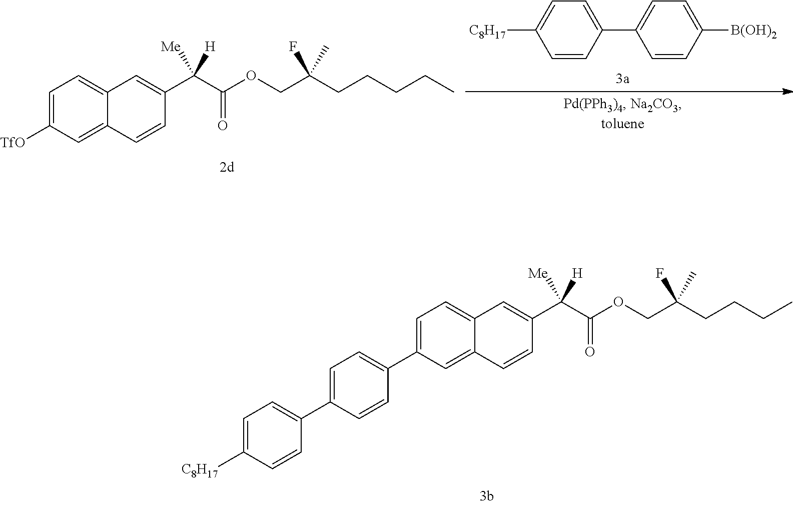

Representative Naproxen-based FLC or NLC chiral components with one ester linkage and one CH₂O linkage at both ends of Naproxen can be readily synthesized by the multistep approach as shown below:

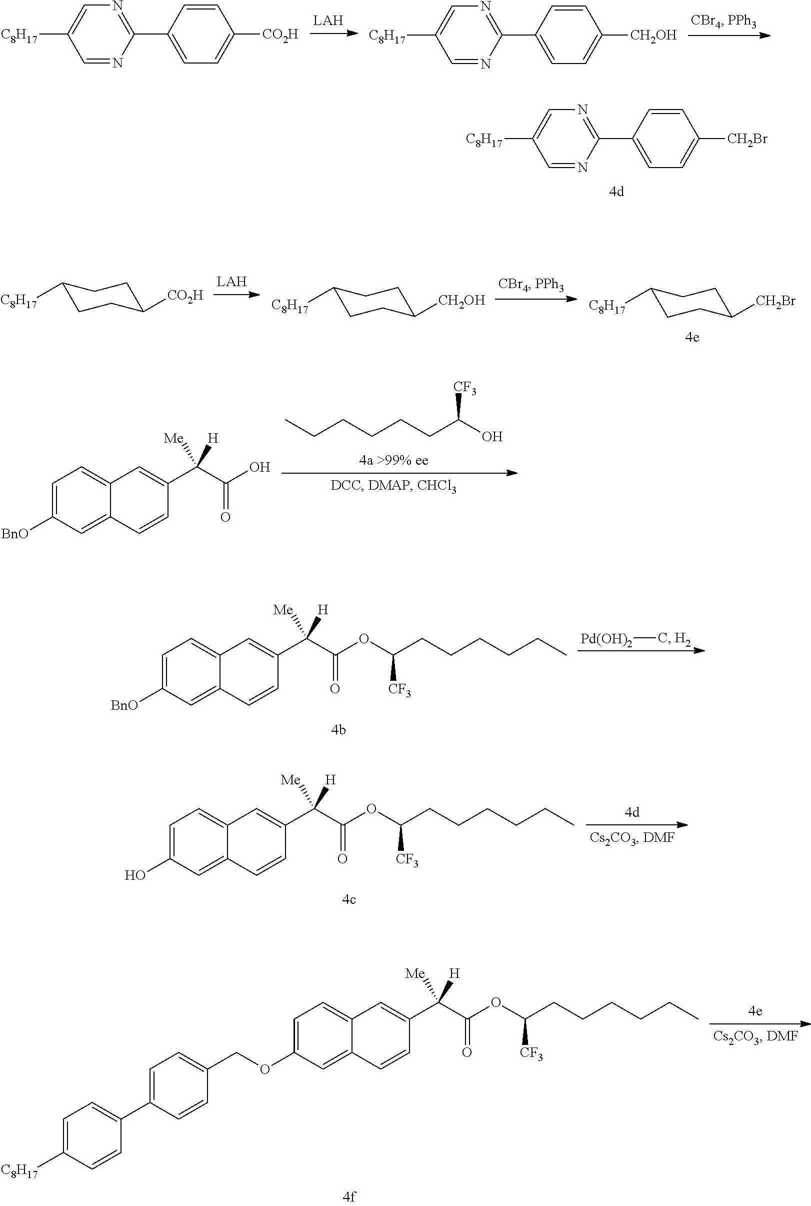

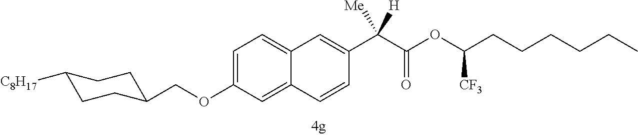

A variety of compounds as illustrated by the general formula I including different cores and tails can be similarly synthesized. Some chiral tails are commercially available or can be readily built from the commercially available chiral starting materials by chiral pool synthesis, and some fluoro-containing chiral compounds can be synthesized according to the literature. (See, U.S. Pat. Nos. 5,380,460; 5,453,218 and 5,585,036). Different chiral epoxides and dihydroxys can be synthesized from a variety of alkenes via SAE (see, Cao, Y. et al., J. Am. Chem. Soc., 1987, 109, 5765-5780) or SAD (see, Kolb, H. C. et al., Chem. Rev., 1994, 94, 2483-2547) reactions. If the ee values of the chiral materials are >99%, they can be directly used for the synthesis of Naproxen-based FLC or NLC chiral dopants. For those with low ee values, chiral resolution using Naproxen or other chiral scaffolds, can be used to improve ee prior to the synthesis of Naproxen-based chiral dopants. If Naproxen should give the undesired sign for the spontaneous polarization, its enantiomer, (−)-(R)-2-(6-methoxynaphthalen-2-yl)propanoic acid could be used to inverse the sign. Silicon- or germanium-containing tails can be synthesized by the literature approaches. (See, U.S. Pat. No. 6,737,124; Zhang et al., Chem. Mater., 2010, 22, 2869-2884; and U.S. Pat. No. 7,425,281 B2). The aromatic cores such as 1,4-phenyl group, 1,4-phenyl group substituted with 1 or 2 halogens, 1,4-phenyl group wherein one or two of the ring carbons are replaced with nitrogen atoms or a thiadiazole ring are commercially available or can be synthesized by the literature approaches. (See, U.S. Pat. Nos. 4,062,798; 3,997,536; 4,452,718; 4,462,923; 4,533,488; 4,657,695; 4,952,699; 5,478,496; and 6,169,184 B1).

Partially Hydrogenated Naproxen FLC and NLC Chiral Components.

The tetrahydronaproxenol should be first prepared from Naproxen using proper chemical transformations. It was reported that 6-hydroxy-2-naphathlene carboxylic acid was selectively reduced to 6-hydroxy-1,2,3,4-tetrahydronaphathlene-2-carboxylic acid using Birch reduction and to 6-hydroxy-5,6,7,8-tetrahydronaphathlene-2-carboxylic acid using selective hydrogenation in the presence of a catalytic amount of 5% Pd—C. (See, EP 549,347 A1). We have found the both methods were reproducible and two partially hydrogenated isomers were obtained in good yields. (See, U.S. Pat. No. 6,703,082).

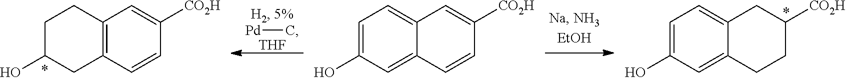

The classic Bamberger method for the reduction of naphathol derivatives using Na and alcohols gave different results. While α-naphathol reduces exclusively to give the phenol, β-naphathol reduces to the alcohol. (See, Bamberger and Lodter Ann. 1895, 288, 74 and Papa et al., J. Org. Chem., 1949, 14, 366-374).

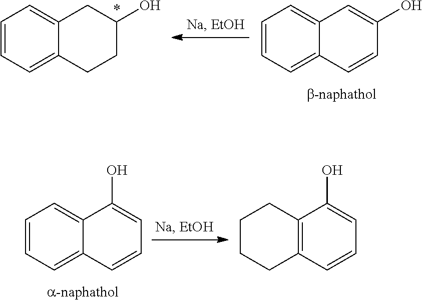 [SMILES: OC1CCc2ccccc2C1 ; Oc1ccc2ccccc2c1 ; Oc1cccc2ccccc12 ; Oc1cccc2c1CCCC2]

Since the Naproxen molecule is absent of the directly linked carboxylic acid group, it is highly likely to reduce Naproxenol to the alcohol using either selective hydrogenation or Birch reduction. We will also attempt to directly reduce Naproxen using both methods to test which ring will be initially reduced in the presence of a methoxy group. The reduction of Naproxenol to the tetrahydronaproxenol generates a carbon chiral center, and thus the resulted alcohol should be a mixture of two diastereomers which could be separated by recrystallization. The alcohol should be a crystalline solid since its molecules contain two hydrogen-bonding functional groups: OH and COOH. Once the tetrahydronaproxenol is obtained, a variety of compounds as illustrated herein can be similarly synthesized by following the procedures herein.

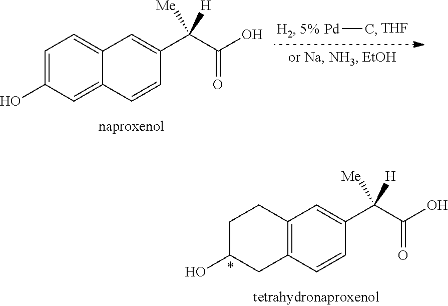

All publications, patents, and patent applications are incorporated herein by reference. While in the foregoing specification this disclosed subject matter has been described in relation to certain specific embodiments thereof, and many details have been set forth for purposes of illustration, it will be apparent to those of ordinary skill in the art that the disclosed subject matter is susceptible to additional embodiments and that certain of the details described herein may be varied considerably without departing from the basic principles of the presently described subject matter.

1. A compound of formula (I): 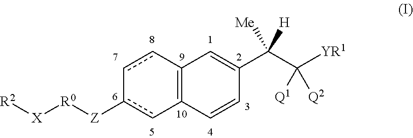
wherein,
R¹ is (C₂-C₂₀)alkyl, (C₂-C₂₀)alkenyl, or (C₂-C₂₀)alkynyl;
R² is (C₂-C₂₀)alkyl, (C₂-C₂₀)alkenyl, or (C₂-C₂₀)alkynyl;
Q¹ and Q² are each hydrogen, or together Q¹ and Q² are oxo (═O) or thioxo (═S);
Z is O—C(═O), C(═O)O, CH₂O, CH₂CH₂, OCH₂, or is absent;
X is oxy (—O—), O—C(═O), C(═O)O, CH₂O, CH₂CH₂, OCH₂, or is absent;
Y is oxy (—O—) or thio (—S—);
R⁰ is an aromatic ring system, a cyclic ring system, a hetero aromatic ring system, a hetero cyclic ring system, or a combination thereof;
each bond represented by - - - - - - is independently optionally present;
any one or more of the aromatic ring system, cyclic ring system, hetero aromatic ring system, hetero cyclic ring system, or combination thereof of R⁰ is optionally substituted on carbon with one or more halo;
any one or more of the (C₂-C₂₀)alkyl, (C₂-C₂₀)alkenyl, or (C₂-C₂₀)alkynyl of R¹ and R² is optionally substituted on carbon with one or more halo; and
any one or more of the (C₂-C₂₀)alkyl, (C₂-C₂₀)alkenyl, or (C₂-C₂₀)alkynyl of R¹ and R² is optionally interrupted with one or more oxy (—O—), thio (—S—), Si(R_{A})(R_{B}) or Ge(R_{A}R_{B}), wherein each R_{A} and R_{B} is independently (C₁-C₆)alkyl or (C₁-C₆)alkenyl.

2. The compound of claim 1, wherein R¹ is a non-racemic chiral group.

3. The compound of claim 1, wherein R¹ is a non-racemic chiral group, selected from: 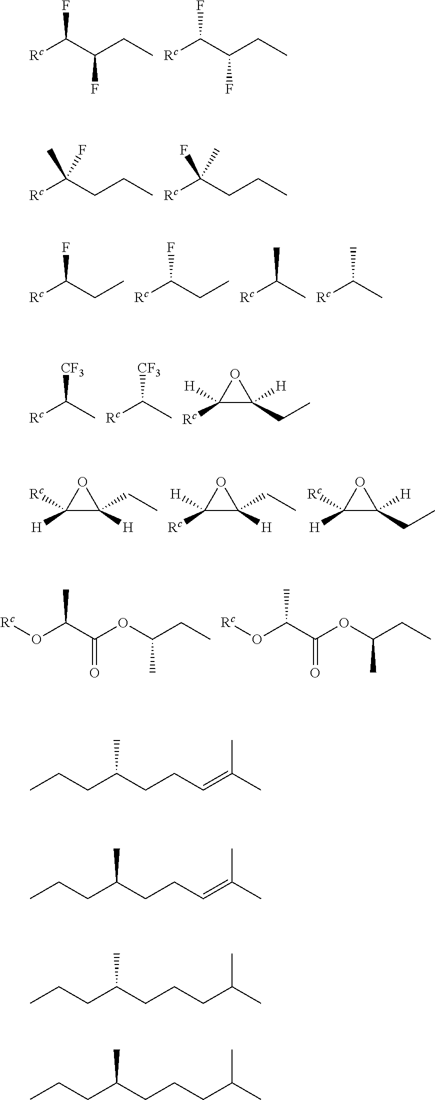
wherein,
Rc is (C₁-C₁₅)alkyl, (C₁-C₁₅)alkenyl, or (C₁-C₁₅)alkynyl, optionally substituted on carbon with one or more halo, and optionally interrupted with one or more oxy (—O—), thio (—S—), Si(R_{A})(R_{B}) or Ge(R_{A}R_{B}), wherein each R_{A} and R_{B} is independently (C₁-C₆)alkyl or (C₁-C₆)alkenyl.

4. The compound of claim 1, wherein R¹ is selected from:

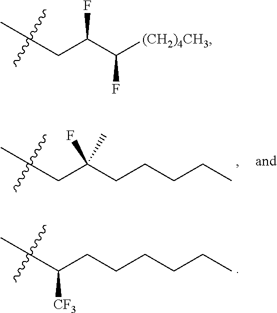

5. The compound of claim 1, wherein R² is a non-racemic chiral group.

6. The compound of claim 1, wherein R² is a non-racemic chiral group, selected from: 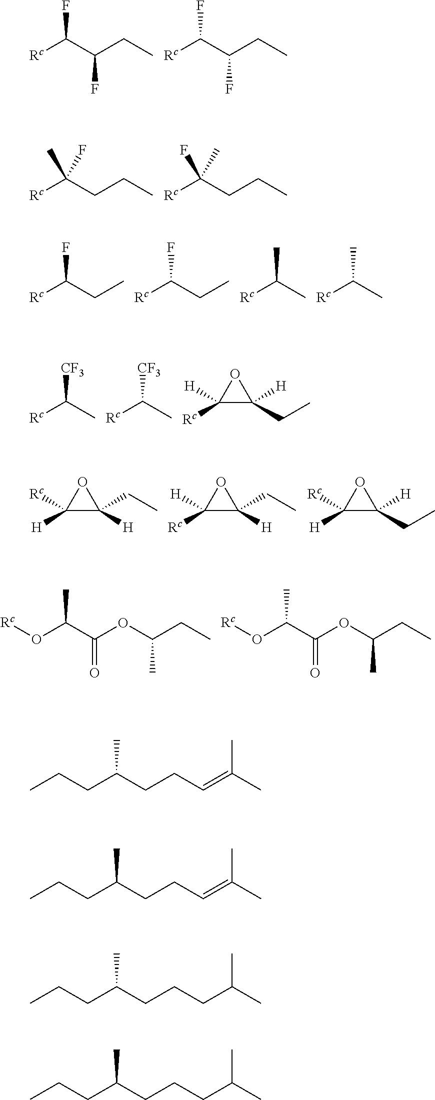
wherein,
Rc is (C₁-C₁₅)alkyl, (C₁-C₁₅)alkenyl, or (C₁-C₁₅)alkynyl, optionally substituted on carbon with one or more halo, and optionally interrupted with one or more oxy (—O—), thio (—S—), Si(R_{A})(R_{B}) or Ge(R_{A}R_{B}), wherein each R_{A} and R_{B} is independently (C₁-C₆)alkyl or (C₁-C₆)alkenyl.

7. The compound of claim 1, wherein R² is selected from:

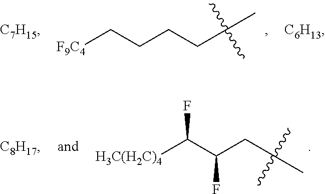

8. The compound of claim 1, wherein R⁰ is
—(R^{x})ₙ-(A)ₐ-(Ph¹)ₗ-(B)_{b}-(Ph²)ₘ-
wherein,
R^{x} is cyclohexyl or cyclohexenyl, optionally substituted on carbon with one or more halo or cyano, and one or two of the methylene (CH₂) groups of the cyclohexyl or cyclohexenyl is optionally independently replaced with oxy (—O—);
n is 0 or 1;
A is oxy (—O—), thio (—S—), CH₂O, OCH₂, CH₂S, SCH₂, CH₂OCO, CH₂CO₂, CH₂CH₂, COS, CSO, COO, OCO, a single bond (absent), a double bond, or a triple bond;
a is 0 or 1;
Ph¹ is 1,4-phenyl, optionally substituted on carbon with one or more halo, and one or two of the ring carbons are optionally independently replaced with nitrogen (N) or a thiadiazole ring;
l is 0 or 1;
B is oxy (—O—), thio (—S—), CH₂O, OCH₂, CH₂S, SCH₂, CH₂OCO, CH₂CO₂, CH₂CH₂, COS, CSO, COO, OCO, a single bond (absent), a double bond, or a triple bond;
b is 0 or 1;
Ph² is 1,4-phenyl, optionally substituted on carbon with one or more halo, and one or two of the ring carbons are optionally independently replaced with nitrogen (N) or a thiadiazole ring;
m is 0 or 1;
when m is 0, then b is also 0; and
at least one of n, a, 1, b and m is 1.

9. The compound of claim 1, wherein R⁰ is an aromatic ring system or a cyclic ring system.

10. The compound of claim 1, wherein R⁰ is phenyl, 3-fluorophenyl, biphenyl, 2,3-difluorophenyl, cyclohexyl, 2-phenylpyrimidine, 2-phenylthiadiazo, or 2-phenylpyridine.

11. The compound of claim 1, wherein the bond represented by - - - - - - between carbon atoms 5 and 6, and the bond represented by - - - - - - between carbon atoms 7 and 8, are each absent.

12. The compound of claim 1, wherein the bond represented by - - - - - - between carbon atoms 5 and 6, and the bond represented by - - - - - - between carbon atoms 7 and 8, are each present.

13. The compound of claim 1, wherein Q¹ and Q² together are oxo (═O).

14. The compound of claim 1, wherein Z is C(═O)O, CH₂CH₂, absent, CH₂O, or OCH₂.

15. The compound of claim 1, wherein X is absent or CH₂O.

16. The compound of claim 1, wherein X is oxy (—O—).

17. The compound of claim 1, which is a compound of the formula:

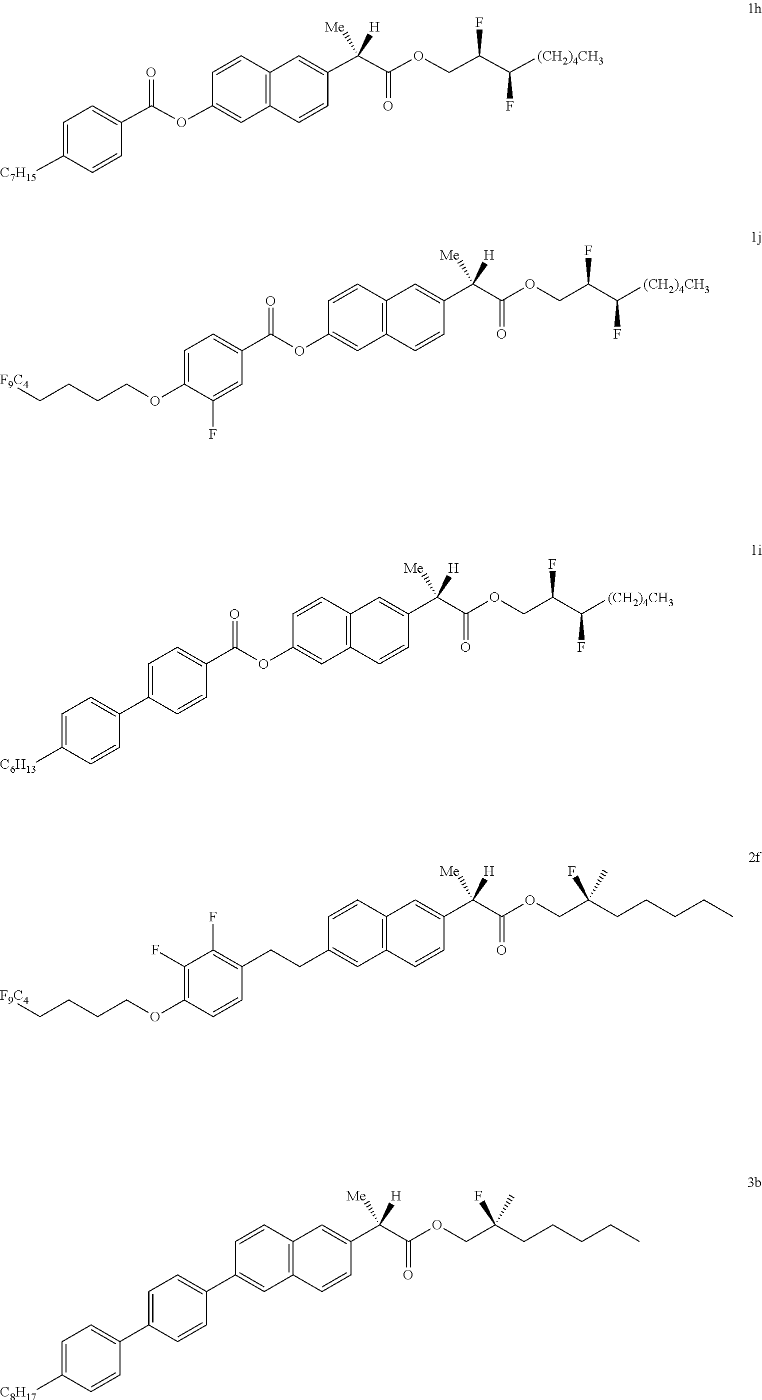

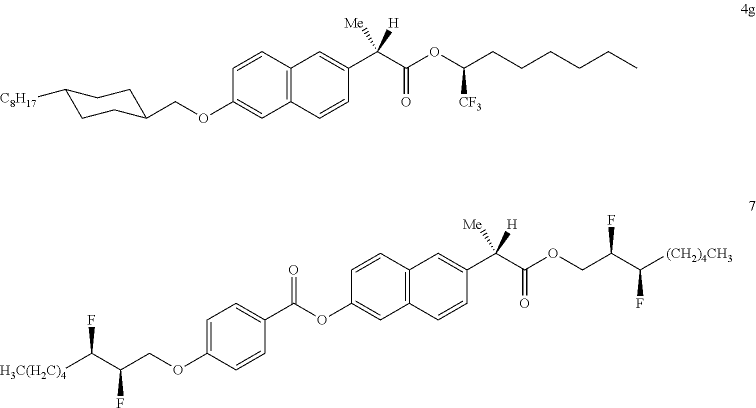

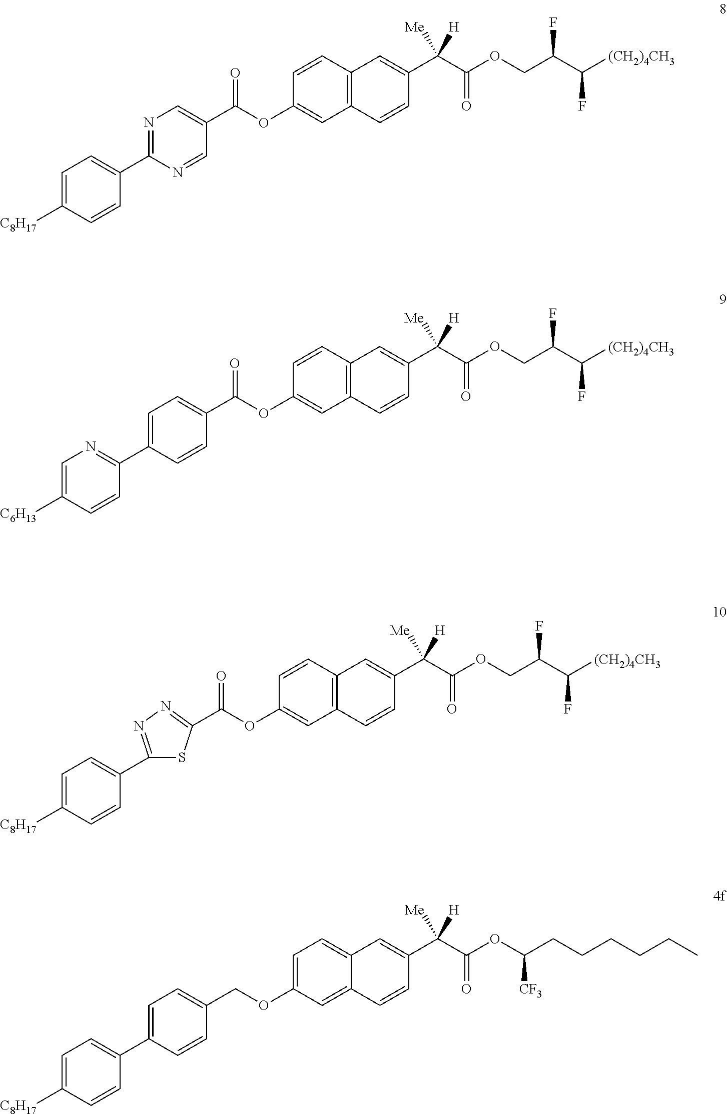

18. A liquid crystal comprising a compound of formula (I): 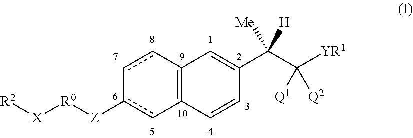
wherein,
R¹ is (C₂-C₂₀)alkyl, (C₂-C₂₀)alkenyl, or (C₂-C₂₀)alkynyl;
R² is (C₂-C₂₀)alkyl, (C₂-C₂₀)alkenyl, or (C₂-C₂₀)alkynyl;
Q¹ and Q² are each hydrogen, or together Q¹ and Q² are oxo (═O) or thioxo (═S);
Z is O—C(═O), C(═O)O, CH₂O, CH₂CH₂, OCH₂, or is absent;
X is oxy (—O—), O—C(═O), C(═O)O, CH₂O, CH₂CH₂, OCH₂, or is absent;
Y is oxy (—O—) or thio (—S—);
R⁰ is an aromatic ring system, a cyclic ring system, a hetero aromatic ring system, a hetero cyclic ring system, or a combination thereof;
each bond represented by - - - - - - is independently optionally present;
any one or more of the aromatic ring system, cyclic ring system, hetero aromatic ring system, hetero cyclic ring system, or combination thereof of R⁰ is optionally substituted on carbon with one or more halo;
any one or more of the (C₂-C₂₀)alkyl, (C₂-C₂₀)alkenyl, or (C₂-C₂₀)alkynyl of R¹ and R² is optionally substituted on carbon with one or more halo; and
any one or more of the (C₂-C₂₀)alkyl, (C₂-C₂₀)alkenyl, or (C₂-C₂₀)alkynyl of R¹ and R² is optionally interrupted with one or more oxy (—O—), thio (—S—), Si(R_{A})(R_{B}) or Ge(R_{A}R_{B}), wherein each R_{A} and R_{B} is independently (C₁-C₆)alkyl or (C₁-C₆)alkenyl.

19. The liquid crystal of claim 18, wherein the liquid crystal is a ferroelectric liquid crystal.

20. A display device comprising the compound of claim 1 or the liquid crystal of claim 18.

21. The display device of claim 20, having at least one of stable memory performance, high contrast ratio, wide angle view, high speed response, and lower power consumption, relative to a comparable device not comprising a compound of claim 1.

22. The display device of claim 20, comprising a ferroelectric liquid crystal composition.

23. The display device of claim 20, wherein the device is a cell phone, a smart phone, a tablet, or a computer display screen.

24. A method of preparing a liquid crystal display device on silicon comprising incorporating a compound of claim 1 or the liquid crystal of claim 18 into a liquid crystal display on silicon by disposing the compound in a liquid crystal, or the liquid crystal, onto a silicon surface.

25. The method of claim 24, wherein the liquid crystal display has at least one of stable memory performance, high contrast ratio, wide angle view, high speed response, and lower power consumption, relative to a comparable device not comprising a compound of claim 1.

26. The method of claim 24, wherein the liquid crystal display device is a ferroelectric liquid crystal display device.
